(19) United States
(12) Patent Application Publication
(10) Pub. No.: US 2024/0091102 A1
(43) Pub. Date: Mar. 21, 2024

(54) Vaccine Product

(71) Applicant: Janssen Vaccines & Prevention B.V., Leiden (NL)

(72) Inventors: Olga Labovitiadi, Degstgeest (NL); Martinus Anne Hobbe Capelle, Sassenheim (NL); Joao Miguel Calado Da Silva Freire, Leiden (NL); Willem Jan Timmer, Oeffelt (NL)

(21) Appl. No.: 17/754,877

(22) PCT Filed: Oct. 16, 2020
(86) PCT No.: PCT/EP2020/079274

§ 371 Date: Apr. 14, 2022

(30) Foreign Application Priority Data
Oct. 16, 2019 (EP) 19386042.6

(51) Int. Cl. A61J 1/14; A61K 39/13; A61K 39/235

(52) U.S. Cl. CPC A61J 1/1468; A61K 39/13; A61K 39/235; A61K 2039/5254

(57) ABSTRACT

The present invention provides a vaccine product comprising a container, wherein the container comprises an internal surface, the internal surface comprising either (i) silicon dioxide, (ii) a polymeric material, or (iii) a surface treated with ethylene oxide; and a vaccine composition within the container in contact with the internal surface, wherein the vaccine composition comprises virus particles.

### CROSS-REFERENCE TO RELATED APPLICATION

This application is a Section 371 of International Application No. PCT/EP2020/079274, filed on Oct. 16, 2020, which published in the English language on Apr. 22, 2021, under International Publication No. WO2021/074423 A2, which claims priority to EP Application No. 19386042.6 filed on Oct. 16, 2019, the disclosures of which are incorporated herein by reference in their entirety.

### FIELD OF INVENTION

The present invention relates to a vaccine product. It further relates to the use of a container for maintaining potency of a vaccine composition.

### BACKGROUND

The stability of biological preparations, such as vaccines, is an important consideration in their formulation. The stability is required to maintain the efficacy and safety of the vaccine composition up to administration to the patient. Any instability can be manifested as a loss of potency of the vaccine, which in turn may lead to an ineffective dose being administered to the patient and so an ineffective vaccination procedure. Consequently, there is a need to provide a vaccine product with improved stability. In order to improve stability, the formulation of the vaccine composition can be optimised. However, it would be useful to provide increased flexibility in formulation design by providing other means for improving the stability of vaccine compositions.

### BRIEF SUMMARY

Provided herein is a formulation or container for maintaining the potency of a vaccine composition.

In one general aspect, the application relates to a method of maintaining potency of a vaccine composition comprising virus particles, comprising:
providing a container that comprises an internal surface, the internal surface comprising at least one of (i) a polymeric material, (ii) silicon dioxide, or (iii) a surface treated with ethylene oxide; and introducing the vaccine composition into the container and in contact with the internal surface.

Another general aspect of the application relates to a method of reducing loss of virus particles from a vaccine composition, comprising:
providing a container that comprises an internal surface, the internal surface comprising at least one of (i) a polymeric material, (ii) silicon dioxide, or (iii) a surface treated with ethylene oxide; and
introducing the vaccine composition into the container and in contact with the internal surface;
wherein the silicon dioxide or the polymeric material are present as part of the internal surface of the container and wherein the treatment with ethylene oxide is applied to an internal surface of the container.

Another general aspect of the application relates to a vaccine product comprising: a container comprising an internal surface comprising at least one of: (i) a polymeric material, (ii) silicon dioxide, or (iii) a surface treated with ethylene oxide; and a vaccine composition within the container in contact with the internal surface, wherein the vaccine composition comprises virus particles.

According to embodiments of the application, the container comprises a borosilicate glass.

According to embodiments of the application, the silicon dioxide or polymeric material is present as a coating.

According to embodiments of the application, the silicon dioxide or polymeric material is present substantially throughout the internal surface of the container.

According to embodiments of the application, the polymeric material is a polysiloxane.

According to embodiments of the application, the container consists essentially of polypropylene.

According to embodiments of the application, the container consists essentially of a cyclic olefin based resin.

According to embodiments of the application, the vaccine composition comprises virus particles having an isoelectric point of from 6 to 8.

According to embodiments of the application, the vaccine composition comprises virus particles having an isoelectric point value within 1 pH unit of the pH of the vaccine composition.

According to embodiments of the application, the vaccine composition has a pH of about 7.

According to embodiments of the application, the virus particles are adenovirus particles.

According to embodiments of the application, the virus particles are inactivated poliovirus or attenuated poliovirus and the inactivated poliovirus or attenuated poliovirus comprises serotype 2, and wherein the inactivated poliovirus or attenuated poliovirus is a Sabin strain.

### BRIEF DESCRIPTION OF THE DRAWINGS

FIG. 1a is a plot that depicts in vitro potency data obtained from D-antigen ELISA for poliovirus type 1.

FIG. 1b is a plot that depicts in vitro potency data obtained from D-antigen ELISA for poliovirus type 2.

FIG. 1c is a plot that depicts in vitro potency data obtained from D-antigen ELISA for poliovirus type 3.

FIG. 1d is a plot that depicts poliovirus concentration obtained by HP-SEC for the drug product containing all three serotypes.

FIG. 2a is a plot that depicts in vitro potency data obtained from D-antigen ELISA for poliovirus type 1.

FIG. 2b is a plot that depicts in vitro potency data obtained from D-antigen ELISA for poliovirus type 2.

FIG. 2c is a plot that depicts in vitro potency data obtained from D-antigen ELISA for poliovirus type 3.

FIG. 2d is a plot that depicts poliovirus concentration obtained by HP-SEC for the drug product containing all three serotypes.

FIG. 3 is a plot that depicts the adenovirus particle recovery for various containers.

FIG. 4a depicts the comparison of adenovirus adsorption for Type 1 non-siliconized vials for different times and orientations.

FIG. 4b depicts the comparison of adenovirus adsorption for Type 1 siliconized vials for different times and orientations.

FIG. 4c depicts the comparison of adenovirus adsorption for siliconized vials treated with ethylene oxide for different times and orientations.

FIG. 4d depicts the comparison of adenovirus adsorption for Type 1 plus (silica coated) vials for different times and orientations.

FIG. 5a is a plot that depicts potency data for vials treated with ethylene oxide, having a siliconized coating and cyclic olefin based vials for poliovirus type 1.

FIG. 5b is a plot that depicts potency data for vials treated with ethylene oxide, having a siliconized coating and cyclic olefin based vials for poliovirus type 2.

FIG. 5c is a plot that depicts potency data for vials treated with ethylene oxide, having a siliconized coating and cyclic olefin based vials for poliovirus type 3.

FIG. 6 is a plot that depicts relative Vp-titer data for siliconized vials and traditional Type 1 glass vials for Ad26 maintained in an upright orientation.

FIG. 7 is a plot that depicts relative Vp-titer data for siliconized vials and traditional Type 1 glass vials for Ad26 maintained in an inverted orientation.

### DETAILED DESCRIPTION

The following description is presented to enable a person of ordinary skill in the art to make and use the various embodiments. Descriptions of specific compositions, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. When used herein the term “comprising” can be substituted with the term “containing” or “including” or sometimes when used herein with the term “having”.

The present invention provides a vaccine product comprising a container, wherein the container comprises an internal surface, the internal surface comprising either (i) silicon dioxide, (ii) a polymeric material, or (iii) a surface treated with ethylene oxide; and a vaccine composition within the container in contact with the internal surface, wherein the vaccine composition comprises virus particles.

The vaccine product may comprise the vaccine composition in a final form that is suitable for administration to the patient. Alternatively, the vaccine composition may require further processing steps in order to produce the vaccine composition in a final form that is suitable for administration to the patient.

It has been found that the vaccine composition being in contact with silicon dioxide, a polymeric material, or a surface treated with ethylene oxide results in a particularly stable vaccine composition. In particular, it has been found that such surfaces allow a greater recovery of the virus particles of a vaccine composition from the container. In this regard, it has not been appreciated previously that, for vaccine compositions comprising virus particles, there is a significant loss of virus particles when the vaccine composition is stored within a container such as a Type 1 glass container. Additionally, it has been found that the vaccine composition being in contact with silicon dioxide, a polymeric material, or a surface treated with ethylene oxide leads to an increased stability of the vaccine composition due to a reduction in the degradation of the vaccine composition with time. Again, this was not previously appreciated. Accordingly, aspects of this invention are the use of silicon dioxide, a polymeric material, or a surface treated with ethylene oxide to increase stability of a vaccine composition, and the use of silicon dioxide, a polymeric material, or a surface treated with ethylene oxide to reduce the degradation of the vaccine composition. Such stability increase and degradation reduction can be appreciated relative to the vaccine composition being stored in a different container, such as a Type 1 glass container.

The container can be any form of container that is suitable for containing a vaccine composition. The container may be in the form of a vial. A vial is particularly useful when the vaccine composition is in the form of a liquid composition.

The container may be of the form of a Blow-Fill-Seal (BFS) container. Such containers are made from a polymeric material, such as polyethylene, that is blow-moulded before being filled with the vaccine composition and then sealed. These containers can be deformed by squeezing in order to assist with the removal of the vaccine composition from the container.

The container may be configured to function as part of a drug delivery device, wherein the drug delivery device is configured to deliver the vaccine composition to the patient. In particular, the container may be able to function as part of a syringe, for example the container may be in the form of a syringe barrel. The container may be attached to a delivery mechanism, such as a needle, for delivering the vaccine composition to a patient. In this way the vaccine composition does not need to be transferred to a further container ahead of delivery avoiding exposure to any further surfaces, and the associated detrimental effects, ahead of delivery to the patient.

In a similar manner, the container may be in the form of a syringe complete with needle. The vaccine composition may be delivered through the needle by squeezing the container, such as the Uniject device and Apiject device.

The container may be any suitable size. For example, when the containers are vials, they may be 2 R vials, 4 R vials, 6 R vials, 8 R vials, 10 R vials or 15 R vials, where vial sizes denoted with R are standard vial sizes conforming to ISO 8362.

The containers may be filled to any suitable degree. For example, the containers may be filled close to capacity of the container, for example over 95% of the capacity of the container may be occupied by the vaccine composition. It has been found that the invention is particularly effective when the vaccine composition fills less than 80% of the vial's capacity, preferably less than 70%, even more preferably less than 60% of the vial's capacity, most preferably 50% or less of the vial's capacity. Without wishing to be bound by theory, it is believed that lower capacity fills result in a higher proportion of interactions with container surface relative to the volume of vaccine composition, thus meaning the invention can have a bigger beneficial effect in these situations.

The container can additionally comprise a lid or stopper to seal the container. This assists in retaining the vaccine composition within the vial and inhibits ingress of unwanted material up to the point when access is required.

The stopper may be a rubber stopper. This is particularly advantageous when a needle is to be utilised to extract the vaccine composition from the container. The stopper may have a coating on its surface, which contacts the vaccine composition within the container. The coating may be a polymeric coating. In particular, the coating may be a polytetrafluoroethylene (PTFE) based coating or an ethylene tetrafluoroethylene (ETFE) based coating. The presence of a polymeric coating on the rubber stopper can assist in maintaining the stability of the vaccine composition.

The container has an internal volume in which the vaccine composition is contained. The internal volume of the container has an internal surface that can contact the vaccine composition when the vaccine composition is present within the container. As noted above, the internal surface comprises silicon dioxide, a polymeric material, or has been treated with ethylene oxide. Having the vaccine composition in contact with these forms of internal surface can contribute to a more stable vaccine composition.

The container may comprise glass. In particular, the container may comprise borosilicate glass. The container may predominantly comprise glass or borosilicate glass. Borosilicate glass is traditionally used as a container material for vaccine compositions due to its chemical resistance and low gas permeability. A particularly preferred form of borosilicate glass is USP/EP JP Type 1 borosilicate glass.

Even though borosilicate glass can be used with the present invention, it has been found that the internal surface of the borosilicate glass should be adjusted in order to improve the stability of the vaccine composition. In particular, it has been found that vaccine compositions containing virus particles that are stored in borosilicate glass containers, such that the vaccine composition is in direct contact with untreated borosilicate glass, exhibit a lower recovery of virus particles of the vaccine composition when the vaccine composition is removed from the container. Accordingly, the present invention requires the internal surface to comprise either silicon dioxide, a polymeric material, or a surface treated with ethylene oxide.

When the internal surface comprises silicon dioxide, the silicon dioxide may be present as a coating that makes up at least part of the internal surface of the container. In particular, the silicon dioxide coating may be present on a borosilicate glass, specifically a Type 1 borosilicate glass. The silicon dioxide coating may comprise silicon dioxide, consist essentially of silicon dioxide, or consist of silicon dioxide.

The silicon dioxide may be present substantially throughout the internal surface of the container. In particular, the silicon dioxide may be present throughout the internal surface of the container apart from any lid or stopper that may be used to seal the container. This ensures that the majority of the surfaces that the vaccine composition may contact within the container are silicon dioxide surfaces. This can improve the stability of the vaccine composition that is present within the container.

The phrase “substantially throughout” refers to at least 80% of the internal surface area of the container, preferably at least 90% of the internal surface area of the container, particularly at least 95% of the internal surface area of the container or all of the internal surface area of the container (excluding the area of any stopper or lid, if present). Typically, substantially throughout refers to at least 95% of the internal surface area of the container.

When the silicon dioxide is present as a coating, it may have a layer thickness in the range of 10 to 500 nanometres, preferably 50 to 300 nanometres, more preferably 100 to 200 nanometres. Such a layer thickness has particularly been found to be effective at improving the stability of the vaccine composition.

The silicon dioxide coating is a non-porous coating ensuring that the vaccine composition does not contact the underlying material.

As noted above, the internal surface may comprise a polymeric material. This polymeric material may be present as a coating.

The polymeric material may be present substantially throughout the internal surface of the container. For example, the polymeric material may be present throughout the internal surface of the container apart from any stopper or lid that may be used to seal the container. Alternatively, the polymeric material may also be present on a stopper or seal that is used to seal the container. Further, the polymeric material may be absent from the area of the container that is in direct contact with the stopper or lid so as to improve the sealing of the container.

The polymeric material may be a non-porous coating ensuring that the vaccine composition does not contact the underlying material.

The polymeric material may be a polysiloxane. The use of the polysiloxane as a surface coating is sometimes referred to as siliconization of the container. The presence of the polysiloxane on the internal surface of the container improves the stability of the vaccine composition.

The siliconization can be achieved by spraying a silicone oil-in-water emulsion onto the internal surface of the container and then heating the container to bake the silicone oil onto the container surface. This is particularly effective with glass containers, such as Type 1 borosilicate glass containers. An example of such a container is a siliconized EZ-fill vial from Nuova Ompi S.r.1., although other containers are available. The siliconization may result in the production of a chemically crosslinked silicone layer.

When the internal surface of the container comprises a polymeric material, the polymeric material may be polypropylene. It has been found that polypropylene is particularly effective at maintaining the stability of the vaccine composition. The polymeric material may alternatively be polyethylene, which has been found to be effective at maintaining the stability of the vaccine composition.

The container utilised in the present invention may consist essentially of polymeric material, such as polypropylene or polyethylene. In other words, the container may be formed exclusively of a polymeric material, such as a homopolymer or copolymer of polypropylene apart, possibly, from any lid or stopper for sealing the container.

When a polymeric material is present on the internal surface of the container, the polymeric material may be a cyclic olefin based resin. In particular, the cyclic olefin based resin may be a cyclic olefin homopolymer or a cyclic olefin copolymer. It has been found that the use of such cyclic olefin based resins is particularly advantageous for maintaining the stability of the vaccine composition.

The container may consist essentially of the cyclic olefin based resin. In this way, the container is formed fully of the compatible material for the internal surface. The container may be fully formed of the cyclic olefin based resin apart from, possibly, any stopper or lid that is used to seal the container. An example of such a cyclic olefin based resin container is the Daikyo Crystal Zenith® vials available from West Pharmaceutical Services, Inc., although other containers are available.

As noted above, the internal surface of the container may be a surface treated with ethylene oxide. It has been found that a surface treated with ethylene oxide leads to a more stable vaccine composition. At least a part of the internal surface of the container may be treated with ethylene oxide. In particular, all of the internal surface may be treated with ethylene oxide, preferably all surfaces of the container are treated with ethylene oxide.

The ethylene oxide treatment may be conducted under standard ethylene oxide based sterilisation conditions. The treatment may be carried out at a temperature of between 30° C. and 60° C., at a relative humidity of above 30%, utilising an ethylene oxide concentration of between 200 and 1000 mg/L and an exposure time of between 2 and 10 hours. The skilled person would be capable of identifying other suitable treatment conditions.

The surface is treated with ethylene oxide prior to the vaccine composition being introduced into the container. There are preferably no intervening processing steps in relation to the internal surface between the treatment with ethylene oxide and the introduction of the vaccine composition into the container. Thus, the surface is treated with ethylene oxide as the last processing step before the vaccine composition is placed in the container. Without wishing to be bound by theory, it is believed that the treatment of the surface with ethylene oxide can result in a polymerised coating being present on the container surface. This is particularly useful when the underlying material is borosilicate glass. In this instance, it is believed that the treatment with ethylene oxide provides a protective layer that inhibits the contact between the vaccine composition and the underlying borosilicate glass.

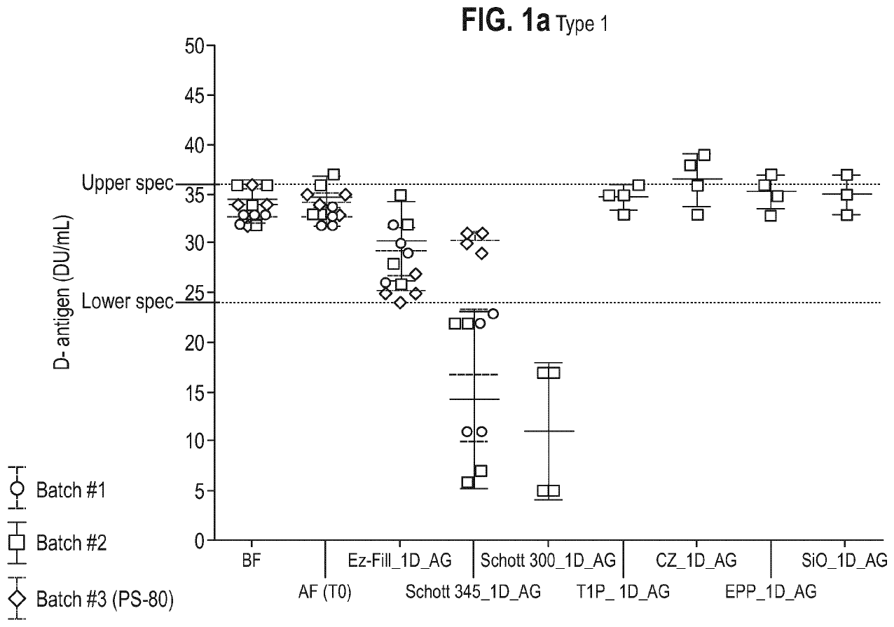

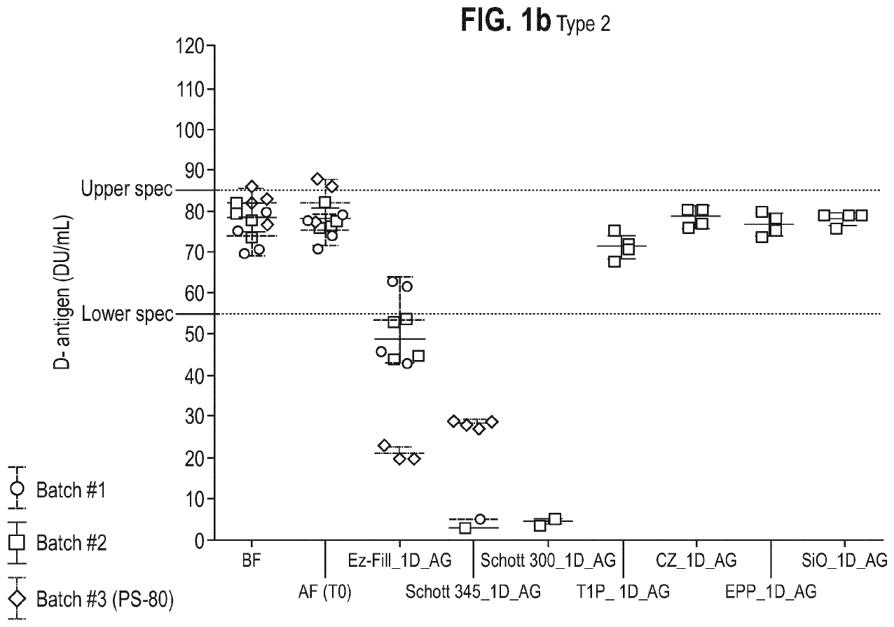

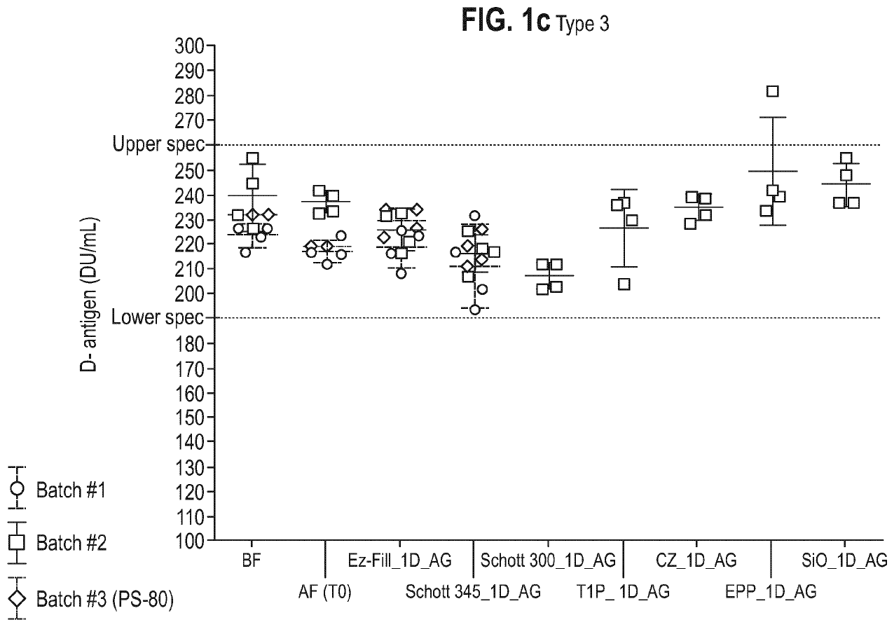

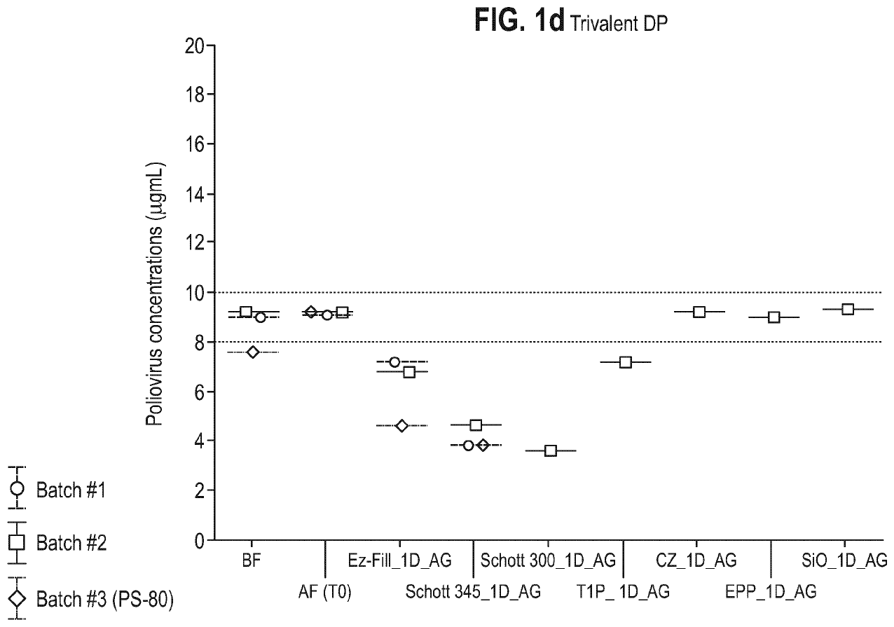

When referring to a surface treated with ethylene oxide herein, the surface is considered to be treated with ethylene oxide if it retains the characteristics of having been treated with ethylene oxide. Such a characteristic includes providing a container that results in an increased stability for the vaccine composition that is introduced into the container. In particular, increased stability for a vaccine composition comprising Sabin inactivated poliovirus serotype 2 as utilised in the present examples and illustrated in FIG. 1.

The container may comprise an internal surface comprising two or more of (i) silicon dioxide, (ii) a polymeric material, and (iii) a surface treated with ethylene oxide. In particular, the internal surface may comprise either (i) silicon dioxide, or (ii) a polymeric material, and also be treated with ethylene oxide.

As noted above, the vaccine composition comprises virus particles. The vaccine composition may comprise one type of virus particle, or a plurality of types of virus particles. The virus particles may include virus particles that are employed as viral vectors for vaccination. In particular, the virus particles may include adenovirus particles employed as viral vectors. The adenovirus may be human adenovirus or a non-human primate adenovirus.

The concentration of virus particles in the vaccine composition may be less than 1×10¹² virus particles per ml. The present invention is particularly effective with relatively low virus particle concentrations as the prevention of virus particle loss and degradation has a more significant effect.

The vaccine composition may comprise virus particles having an isoelectric point of from 6 to 8. It has particularly been found that such virus particles benefit when utilised with the present invention. Preferably the vaccine composition comprises virus particles having an isoelectric point of from 6 to 7.

The isoelectric point of the virus particles is measured using a capillary isoelectric focussing-whole column imaging detection method. The method is described in Thomassen et al Anal. Chem. 2013, 85, 6089-6094: “Isoelectric point determination of live polioviruses by capillary isoelectric focussing with whole column imaging detection”, which is incorporated herein by reference.

Without wishing to be bound by theory, it is believed that virus particles having an isoelectric point value near the pH of the vaccine composition are particularly vulnerable to instability and thus a drop in potency during storage. The present invention is therefore particularly advantageous in these situations. For example, the invention is particularly advantageous when the vaccine composition comprises virus particles that have an isoelectric point value within one pH unit of the pH of the vaccine composition, preferably within 0.5 pH units of the vaccine composition, more preferably within 0.3 pH units of the pH of the vaccine composition or 0.2 pH unit of the pH of the vaccine composition.

The present invention has been found to be particularly effective when the vaccine composition has a pH of about 7. The pH of the vaccine composition may be 7.

The present invention has particularly been found to be effective with virus particles that are RNA virus particles. In particular, the vaccine product has particularly been found to be effective where the virus particles are inactivated poliovirus or attenuated poliovirus. The inactivated poliovirus or attenuated poliovirus may comprise serotypes 1, 2 or 3. The present invention has been found to be particularly beneficial for serotypes 1 and 2, and especially serotype 2.

The poliovirus is preferably a Sabin strain. The invention has particularly been found to be effective with this strain of poliovirus.

The present invention has also particularly been found to be effective with virus particles that are DNA virus particles. In particular, the vaccine product has particularly been found to be effective where the virus particles are adenovirus particles. The adenovirus may be utilised as a viral vector for the vaccine, such as the Ad26.preF based respiratory syncytial virus vaccine.

The vaccine composition may be targeted at poliovirus, respiratory syncytial virus, human immunodeficiency viruses, or coronaviruses (such as COVID-19).

The vaccine composition is preferably in the form of a liquid. The vaccine composition may be in the form of a powder, such as a lyophilised or spray-dried powder. In either case, the presence of silicon dioxide, a polymeric material, or a surface treated with ethylene oxide can be beneficial for the vaccine composition.

The present invention further relates to a method for producing a vaccine product, the method comprising the steps of providing a container, wherein the container comprises an internal surface, the internal surface comprising at least one of (i) silicon dioxide, (ii) a polymeric material, or (iii) a surface treated with ethylene oxide; and introducing a vaccine composition into the container to produce the vaccine product, such that the vaccine composition is in contact with the internal surface, and wherein the vaccine composition comprises virus particles.

The method may further comprise a step of treating the internal surface with ethylene oxide. This step has the benefit of providing a container that is capable of maintaining the potency of the vaccine composition.

The present invention further relates to the use of a container for maintaining potency of a vaccine composition, wherein the container comprises an internal surface, the internal surface comprising either (i) silicon dioxide, (ii) a polymeric material, or (iii) a surface treated with ethylene oxide; and wherein the vaccine composition is within the container and in contact with the internal surface, wherein the vaccine composition comprises virus particles.

Further, the present invention provides the use of (i) silicon dioxide, (ii) a polymeric material, or (iii) treatment with ethylene oxide to maintain the potency of a vaccine composition, wherein the silicon dioxide or the polymeric material are present on the internal surface of a container and wherein the treatment with ethylene oxide is applied to the internal surface of a container. The vaccine composition can then be present within the container and be in contact with the internal surface. As noted herein, the vaccine composition comprises virus particles.

Additionally, the present invention provides the use of (i) silicon dioxide, (ii) a polymeric material, or (iii) treatment with ethylene oxide to reduce loss of virus particles from a vaccine composition, wherein the silicon dioxide or the polymeric material are present as part of an internal surface of a container and wherein the treatment with ethylene oxide is applied to an internal surface of a container, in particular to reduce loss of virus particles from a vaccine composition by adsorption to the internal surface of the container. In other words, this use is a use to increase the amount of virus particles of the vaccine composition that can be recovered from the container, for example, recovered from the container within 44 hours of first being placed in the container, preferably within 22 hours of first being placed in the container, or most preferably within 1 hour of first being placed in the container relative to the loss of virus particles in an equivalent-sized Type 1 standard bulk vial, in particular a Schott Type 1 borosilicate bulk vial sterilised by depyrogenation at 300° C. This recovery may be measured by the vp-qPCR approach described herein.

In this regard, the present invention may be used to reduce a significant loss of virus particles. A significant loss of virus particles may be a loss that is appreciated by the skilled person as being undesirably detrimental for the vaccine composition. A significant loss may be a recovery of virus particles from the container of less than 95% of the virus particles that were originally introduced into the container, preferably less than 90%, more preferably less than 88%, even more preferably less than 85% or most preferably less than 82%. This loss is measured by the vp-qPCR approach described herein after the vaccine composition has been in the container for 48 hours at 5° C.

Further, a reduction in significant loss of virus particles may be defined by the loss of virus particles associated with using the container relative to the loss of virus particles in an equivalent-sized Type 1 standard bulk vial, in particular a Schott Type 1 borosilicate bulk vial sterilised by depyrogenation at 300° C. In this sense, the reduction in significant loss of viral particles may be a relative reduction in loss of virus particles of 10% or more, 20% or more, 30% or more, 40% or more, preferably 50% or more, more preferably 70% or more, even more preferably 80% or more, most preferably 90% or more. This loss is measured by the vp-qPCR approach described herein after the vaccine composition has been in the container for 48 hours at 5° C.

As noted above, it has been found that the presence of particular internal surfaces within a container enable the maintenance of the potency of a vaccine composition, and thus improves the stability of the vaccine composition. The particular details relating to the container and the vaccine composition given herein can be utilised with any aspect of the present invention. The potency of the vaccine composition may refer to the recovery of virus particles from the container where an improved maintenance of potency is demonstrated by a higher recovery of virus particles from the container. The recovery of virus particles from the container is a measure of total amount of virus particles that are present in the vaccine composition when the vaccine composition is emptied from the container relative to the total amount of virus particles initially introduced into the container.

The following list of embodiments forms part of the description. These embodiments may be combined in any compatible combination beyond those expressly given below. They can also be combined with any other compatible features described herein.

Embodiment 1. Use of (i) silicon dioxide, (ii) a polymeric material, or (iii) treatment with ethylene oxide to maintain the potency of a vaccine composition, wherein the silicon dioxide or the polymeric material are present as part of an internal surface of a container and wherein the treatment with ethylene oxide is applied to an internal surface of a container.

Embodiment 2. Use of a container for maintaining potency of a vaccine composition, wherein the container comprises an internal surface, the internal surface comprising at least one of (i) silicon dioxide, (ii) a polymeric material, or (iii) a surface treated with ethylene oxide; and wherein the vaccine composition is within the container and in contact with the internal surface, wherein the vaccine composition comprises virus particles.

Embodiment 3. Use of (i) silicon dioxide, (ii) a polymeric material, or (iii) treatment with ethylene oxide to reduce loss of virus particles from a vaccine composition, wherein the silicon dioxide or the polymeric material are present as part of an internal surface of a container and wherein the treatment with ethylene oxide is applied to an internal surface of a container.

Embodiment 4. A vaccine product comprising a container, wherein the container comprises an internal surface, the internal surface comprising at least one of (i) silicon dioxide, (ii) a polymeric material, or (iii) a surface treated with ethylene oxide; and a vaccine composition within the container in contact with the internal surface, wherein the vaccine composition comprises virus particles.

Embodiment 5. The use of any one of embodiments 1 to 3, or the vaccine product of embodiment 4, wherein the container comprises a borosilicate glass.

Embodiment 6. The use of any one of embodiments 1, 2, 3 or 5, or the vaccine product of embodiment 4 or 5, wherein the silicon dioxide is present as a coating.

Embodiment 7. The use of any one of embodiments 1, 2, 3, 5 or 6, or the vaccine product of any one of embodiments 4 to 6, wherein the silicon dioxide is present substantially throughout the internal surface of the container.

Embodiment 8. The use of any one of embodiments 1, 2, 3 or 5 to 7, or the vaccine product of any one of embodiments 4 to 7, wherein the polymeric material is present as a coating.

Embodiment 9. The use of any one of embodiments 1, 2, 3 or 5 to 8, or the vaccine product of any one of embodiments 4 to 8, wherein the polymeric material is present substantially throughout the internal surface of the container.

Embodiment 10. The use of embodiment 8 or embodiment 9, or the vaccine product of embodiment 8 or embodiment 9, wherein the polymeric material is a polysiloxane.

Embodiment 11. The use of embodiment 1, 2, or 3, or the vaccine product of embodiment 4, wherein the polymeric material is polypropylene.

Embodiment 12. The use of embodiment 11, or the vaccine product of embodiment 11, wherein the container consists essentially of polypropylene.

Embodiment 13. The use of embodiment 1, 2, or 3, or the vaccine product of embodiment 4, wherein the polymeric material is a cyclic olefin based resin.

Embodiment 14. The use of embodiment 13, or the vaccine product of embodiment 13, wherein the container consists essentially of the cyclic olefin based resin.

Embodiment 15. The use of embodiment 13 or embodiment 14, or the vaccine product of embodiment 13 or embodiment 14, wherein the cyclic olefin based resin is a cyclic olefin copolymer.

Embodiment 16. The use of any one of embodiments 1, 2, 3 or 5 to 15, or the vaccine product of any one of embodiments 4 to 15, wherein the treatment with ethylene oxide has been carried out substantially throughout the internal surface.

Embodiment 17. The use of any one of embodiments 1, 2, 3 or 5 to 16, or the vaccine product of any one of embodiments 4 to 16, wherein the vaccine composition comprises virus particles having an isoelectric point of from 6 to 8.

Embodiment 18. The use of embodiment 17, or the vaccine product of embodiment 17, wherein the vaccine composition comprises virus particles having an isoelectric point of from 7 to 8.

Embodiment 19. The use of any one of embodiments 1, 2, 3 or 5 to 18, or the vaccine product of any one of embodiments 4 to 18, wherein the vaccine composition comprises virus particles having an isoelectric point value within 1 pH unit of the pH of the vaccine composition.

Embodiment 20. The use of embodiment 19, or the vaccine product of embodiment 19, wherein the vaccine composition comprises virus particles having an isoelectric point value within 0.5 pH units of the pH of the vaccine composition.

Embodiment 21. The use of embodiment 20, or the vaccine product of embodiment 20, wherein the vaccine composition comprises virus particles having an isoelectric point value within 0.3 pH units of the pH of the vaccine composition.

Embodiment 22. The use of any one of embodiments 1, 2, 3 or 5 to 21, or the vaccine product of any one of embodiments 4 to 21, wherein the vaccine composition has a pH of about 7.

Embodiment 23. The use of any one of embodiments 1, 2, 3 or 5 to 22, or the vaccine product of any one of embodiments 4 to 22, wherein the virus particles are RNA virus particles.

Embodiment 24. The use of embodiment 23, or the vaccine product of embodiment 23, wherein the virus particles are inactivated poliovirus or attenuated poliovirus.

Embodiment 25. The use of embodiment 24, or the vaccine product of embodiment 24, wherein the inactivated poliovirus or attenuated poliovirus comprises serotype 2.

Embodiment 26. The use of embodiment 24 or embodiment 25, or the vaccine product of embodiment 24 or embodiment 25, wherein the inactivated poliovirus or attenuated poliovirus comprises serotype 1.

Embodiment 27. The use of any one of embodiments 24 to 26, or the vaccine product of any one of embodiments 24 to 26, wherein the inactivated poliovirus or attenuated poliovirus is a Sabin strain.

Embodiment 28. The use of any one of embodiments 1, 2, 3 or 5 to 22, or the vaccine product of any one of embodiments 4 to 22, wherein the virus particles are DNA virus particles.

Embodiment 29. The use of embodiment 28, or the vaccine product of embodiment 28, wherein the virus particles are adenovirus particles.

Embodiment 30. The use of embodiment 29, or the vaccine product of embodiment 29, wherein the adenovirus particles comprise adenovirus serotype 26.

Embodiment 31. The use of any one of embodiments 1, 2, 3 or 5 to 30, or the vaccine product of any one of embodiments 4 to 30, wherein the virus particles are employed as viral vectors.

Embodiment 32. The use of any one of embodiments 1, 2, 3 or 5 to 31, or the vaccine product of any one of embodiments 4 to 31, wherein the vaccine composition is in the form of a liquid.

Embodiment 33. A method for producing a vaccine product, the method comprising the steps of providing a container, wherein the container comprises an internal surface, the internal surface comprising at least one of (i) silicon dioxide, (ii) a polymeric material, or (iii) a surface treated with ethylene oxide; and introducing a vaccine composition into the container to produce the vaccine product, such that the vaccine composition is in contact with the internal surface, and wherein the vaccine composition comprises virus particles.

Embodiment 34. The method of embodiment 33, further comprising the step of treating the internal surface of the container with ethylene oxide to provide the surface treated with ethylene oxide.

### EXAMPLES

The following examples are offered to illustrate but not to limit the invention. One of skill in the art will recognize that the following procedures may be modified using methods known to one of ordinary skill in the art.

### Example 1. The Potency and Stability of Sabin Inactivation Poliovirus Vaccine in Different Containers

Three batches of Sabin inactivated poliovirus vaccine (sIPV) were prepared. These were filled into six different containers. The details for the containers are as follows.

| Nr* | Primary packaging | Material and manufacturer |
|---|---|---|
| 1 | EZ-fill | Type 1 borosilicate, ready to use, |
| | | pre-sterilized vial (Nuova Ompi) |
| 2 | Schott | Type 1 borosilicate, bulk (Schott), |
| | (345 and 300) | sterilized by depyrogenation (dry |
| | | heat) at lab scale, at 345° C. and |
| | | 300° C., respectively |
| 3 | T1P | Type 1 plus borosilicate quartz-like |
| | | coating, bulk (Schott) |
| 4 | CZ | Plastic vial, cyclic olefin copolymer |
| | | (COC), (Daikyo-West) |
| 5 | SiO | Silicone coated glass vial, ready to use, |
| | | pre-sterilized vial (Nuova Ompi) |
| 6 | EPP | Eppendorf tube, plastic polypropylene |
| | | (Millipore) |

All tested containers, were placed at representative stress conditions (e.g. agitation and 2-8° C.) and analyzed with the D-antigen enzyme-linked immunosorbent assay (ELISA) for assessing in-vitro potency and high pressure size exclusion chromatography for poliovirus concentration.

FIGS. 1a-d shows the data after 24 hours of agitation at 200 rpm at ambient temperature (15-25° C.), for all the containers tested. In-process monitoring samples taken before filtration (BF) and after filtration (AF), kept at 2-8° C., were used as representative controls. The D-antigen analysis was performed in a 2×2 assay format.

The D-Antigen data revealed that glass vials (Type 1 borosilicate) represented by the Schott vials sterilised at 300° C. and 345° C. (Schott 345 and Schott 300) caused the biggest potency drops. It was observed that the drop in potency was strain-specific, where serotype 2 had the largest drop and serotype 3 the smallest.

Such a severe drop was not observed with the EZ-Fill vials of Nuova Ompi (EZ-Fill). These had an internal surface treated with ethylene oxide. Also, the Type 1 Plus vial (TiP), which is a borosilicate glass vial with an internal surface coated with silicon dioxide showed comparable in vitro potency to the EZ-fill vial. However, Type 1 Plus showed the highest propensity of aggregation after 24 hours of agitation when analysed with dynamic light scattering compared to all other materials tested.

Siliconized EZ-Fill glass vials (SiO) and the cyclic olefin based vials (CZ) were among the containers where no immediate adsorption was observed, as analysed by D-antigen ELISA (potency) and high pressure size exclusion chromatography, HP-SEC, (poliovirus total protein concentration). Eppendorfs made out of polyprolylene material (EPP), showed comparable results to the cyclic olefin based vials and the silicon dioxide coated vials, after 24 hours agitation at ambient temperature (15-25° C.) at 200 rpm.

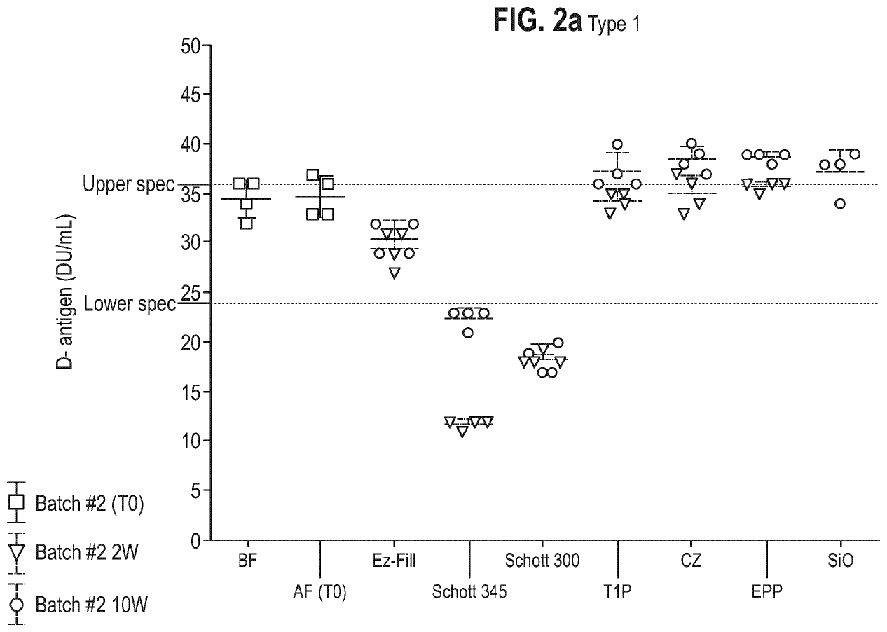

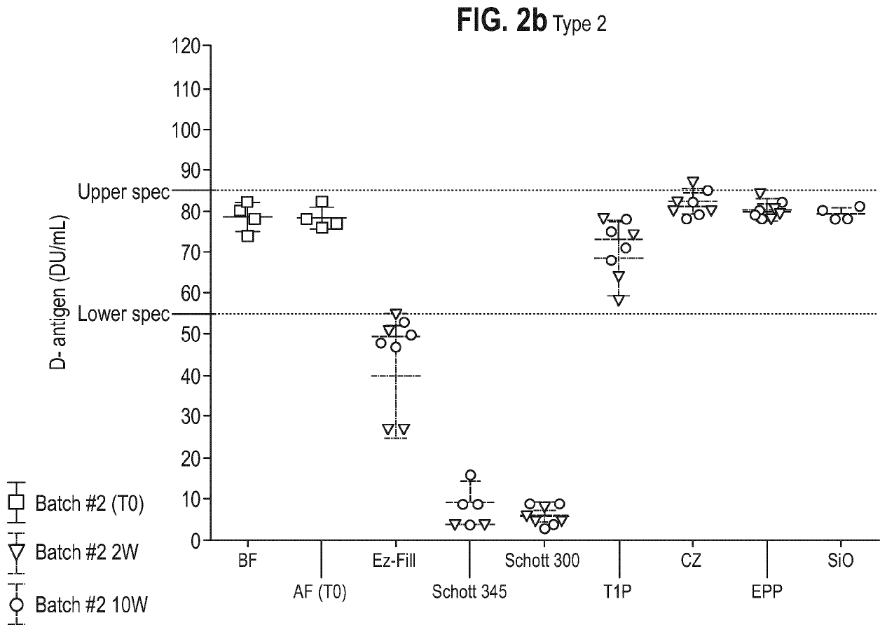

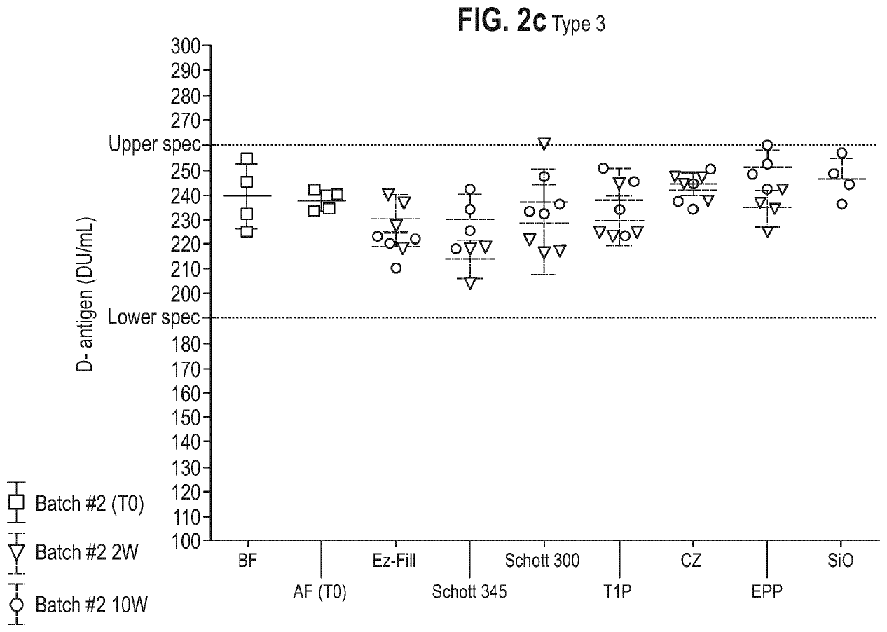

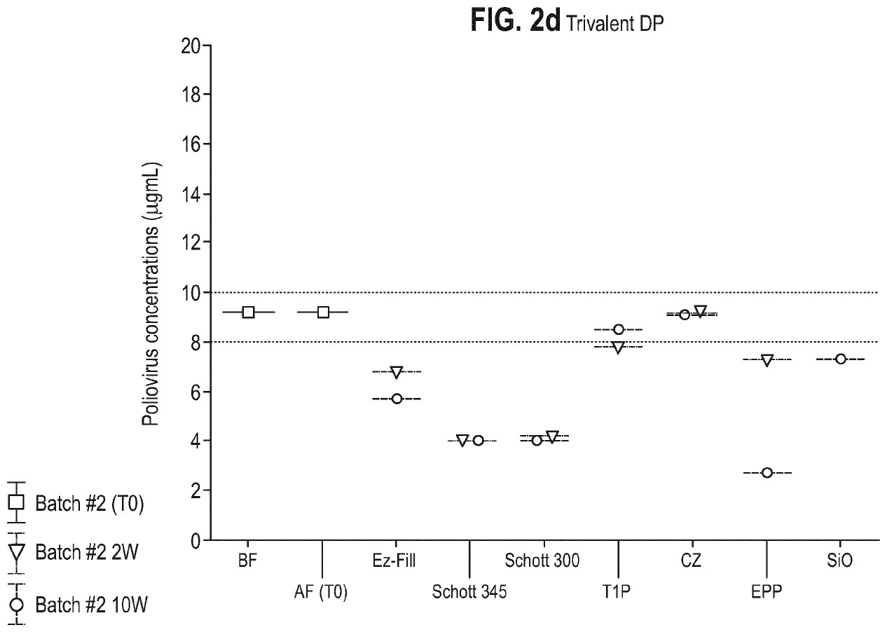

Samples for sIPV Batch #2 were kept at the storage temperature for inactivated polio vaccine (2-8° C.) within all the tested containers and analyzed at approximately 2 and 10 weeks, with the potency assays (D-antigen Elisa) and HP-SEC. The data presented in FIGS. 2a-d demonstrate that the potency of the sIPV after 10 weeks (10 W) of storage at 2-8° C. in some tested containers (TiP, CZ, EPP and SiO) remains in the same levels compared to the initial values (TO). In the case of serotype 1, there is a slight elevation in the in vitro potency after 10 weeks of storage, compared to the initial values and values after two weeks (2 W). This can be related to assay variability or assay performance.

In conclusion, the above data indicate that cyclic olefin vial (CZ) and the siliconized borosilicate glass vial (SiO) are the best performing containers for all three serotypes of the sIPV for up to 10 weeks of testing at 2-8° C. Further, the ethylene treated vials (EZ-Fill) and the silicon dioxide coated vial (TiP) showed improved stability for the vaccine compositions relative to the borosilicate glass vial without such an internal surface treatment (Schott 345 and Schott 300).

The beneficial effect on the potency was most pronounced for serotype 2 and least pronounced for serotype 3. The isoelectric points of the different serotypes of Sabin poliovirus is given below.

| Serotype | Isoelectric point |
|---|---|
| 1 | 7.42 ± 0.07 |
| 2 | 7.18 ± 0.08 |
| 3 | 6.34 ± 0.03 |

This shows that the container choice was most effective at increasing stability for the poliovirus type with an isoelectric point of 7.18 and least beneficial for the poliovirus type with an isoelectric point of 6.34. This can be compared to the vaccine composition pH of 6.9±0.5 showing that the container choice was most effective at increasing the stability of the poliovirus type closest to the vaccine composition pH.

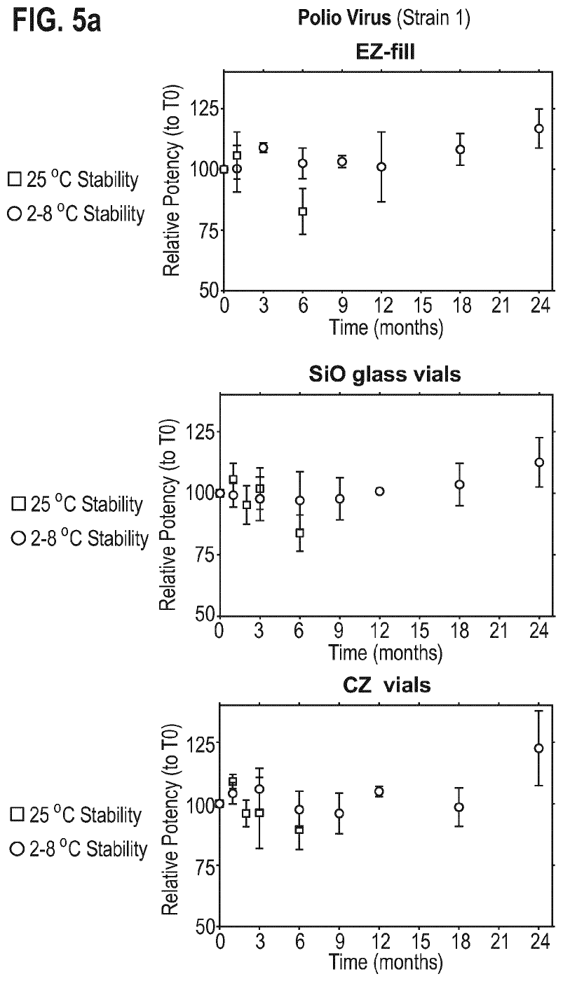

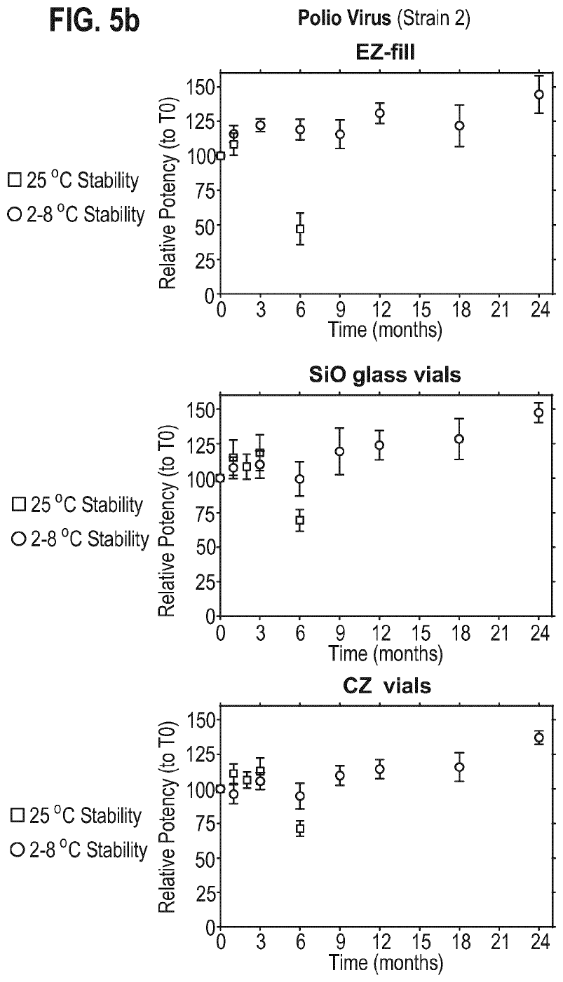

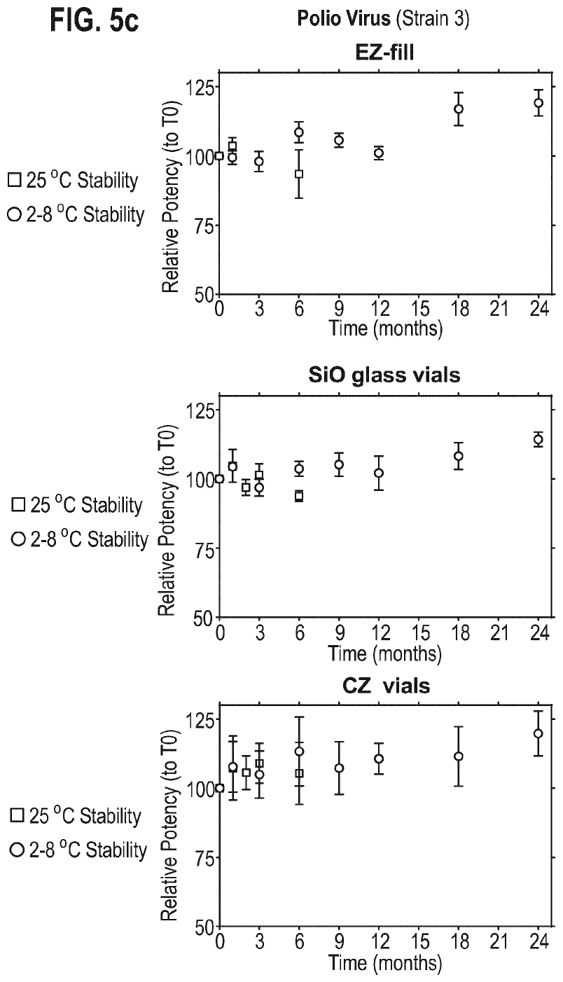

The long term stability of the ethylene oxide treated vials (EZ-fill), the siliconized vials (SiO glass vials), and the cyclic olefin based vials (CZ vials) over the course of up to 24 months is demonstrated by FIGS. 5a-c that reports normalised D-antigen data. For each of these vials effective maintenance of potency is demonstrated when the vials are stored at 2-8° C. for each of the serotypes of poliovirus.

The effect of containers on vaccine compositions was further analysed using a vaccine composition containing adenovirus utilised as a viral vector. The vaccine composition comprised Ad26.RSV.preF.

The amount of virus particles that could be readily recovered from the containers was measured using virus particle quantitative polymerase chain reaction (vp-qPCR) or reverse phase high performance liquid chromatography (RP-HPLC).

The vp-qPCR was developed for the quantification of adenovirus particles using primers that target a 100 bp region of the CMV promoter of the transgene cassette present within the adenovirus vector. Briefly, this qPCR method relies on the exonuclease activity of Taq polymerase, which results in degradation of a specific fluorescent probe annealed in the middle of the 100 bp amplicon. The probe is covalently linked to a light emitter and a quencher, and its degradation frees the emitter from the quencher with a consequent fluorescence emission proportional to the amount of template. Quantitative values are obtained from the threshold cycle (Ct), the cycle at which an increase in fluorescence signal exceeds a threshold value. The threshold for detection of DNA-based fluorescence is set slightly above background. The number of cycles at which the fluorescence exceeds the threshold is called the threshold cycle (Ct) or, according to the MIQE guidelines, quantification cycle (Cq) (Bustin S A et al. The MIQE guidelines: minimum information for publication of quantitative real-time PCR experiments. Clin Chem. 2009 April; 55(4):611-22). During the exponential amplification phase, the target DNA sequence doubles every cycle. For example, a DNA sample of which the Ct precedes that of another sample by three cycles contained 23=8 times more template. Consequently, a higher Ct value represents a lower amount of target DNA and a lower Ct value represents a high availability of target DNA. Absolute quantification can be performed by comparing a standard curve generated by a serial dilution of a stock adenovirus of which the concentration has been determined by the optical density at 260 nm (OD₂₆₀). The Ct values of the test material are plotted against the Ct values of the standard curve, which generates an accurate and precise number of vector particles.

RP-HPLC separates components of a mixture by using a variety of chemical interactions between the sample, the mobile phase (a buffer or solvent) and the stationary phase (a chromatographic packing material in a column). A high-pressure pump moves the mobile phase through the column and a detector shows the retention times (tR; time between sample injection and the appearance of the peak maximum) of the molecules using UV absorbance detection at 280 nm. The separation of RP-HPLC is based on differences in hydrophobicity. The non-polar stationary phase is made up of hydrophobic alkyl chains (chain lengths: C4, C8 and C18). The polar mobile phase is water with 0.1% trifluoroacetic (TFA). Compounds that bind to the columns are eluted using an increasing concentration of acetonitrile with 0.1% TFA. In general, an analyte with a larger hydrophobic surface area has a longer retention time, whereas the presence of polar groups reduce retention time. A typical adenoviral RP-HPLC profile consists of 10 or 14 proteins, including core protein (VII), penton base (III) and hexon (II).

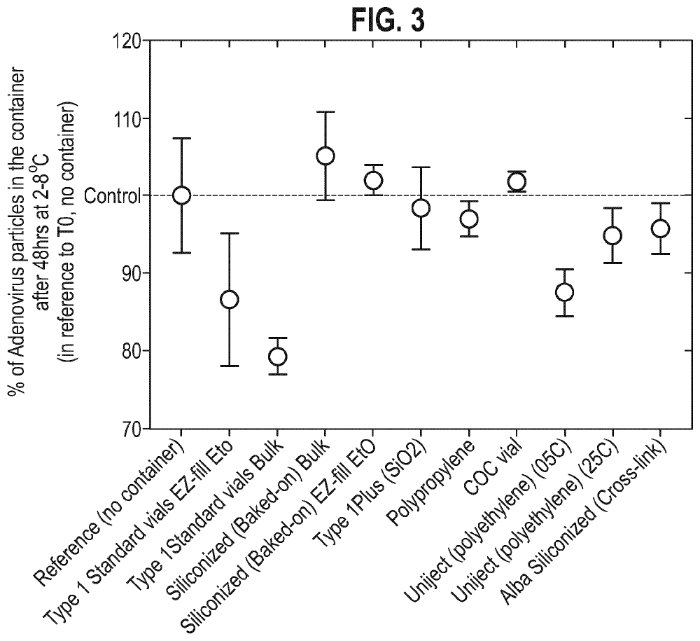

The results for the variety of containers is given in FIG. 3. In this plot the amount of adenovirus particles that were recovered from each container is plotted relative to the amount of particles initially present before introduction into the container, given as “Reference (no container)”. All of the data reported in FIG. 3 is determined by vp-qPCR apart from the determination of virus content for the cyclic olefin copolymer container from Daikyo-West (COC) and the Uniject packaging that is made from polyethylene (Uniject 05C and Uniject 25 C, which were maintained at 2-8° C. and 25° C., respectively). The data in FIG. 3 gives the virus recovery after two days in contact with each container, apart from the “Alba Siliconized (Cross-link)”that was measured after 6 weeks in contact with the container but is included in FIG. 3 for reference.

As seen in FIG. 3, the “Type 1 Standard vials Bulk” from Schott, which have no coating on the internal surface have the lowest recovery of adenovirus particles from the container. A treatment with ethylene oxide, as for the “Type 1 Standard vials EZ-fill EtO”, which are Type 1 borosilicate glass vials supplied from Nuova Ompi, appears to improve the recovery of adenovirus particles, consistent with the results for the poliovirus vaccine composition reported above.

The containers formed from polypropylene, polyethylene (Uniject) and cyclic olefin copolymer (COC) all have improved adenovirus particle recovery. Further, the presence of a siliconisation to provide a polysiloxane layer also improves the adenovirus recovery. Such a polysiloxane layer is present for “Siliconized (Baked-on) Bulk” containers from Nuova Ompi, “Siliconized (Baked-on) EZ-fill EtO” containers from Nuova Ompi and “Alba siliconized (Cross-link)” containers from Nuova Ompi. The presence of a silicon dioxide layer in the “Type 1 Plus (SiO₂)” containers, which are Type 1 borosilicate vials with a quartz-like coating from Schott, also improves adenovirus particle recovery.

In order to detect whether Ad26 protein components were adsorbing to the surface of glass vials, gold nanoparticle protein staining solution was used as a staining solution. The method was as follows as follow:
Remove Ad26 drug product solution from the vials (store for future analysis if planned);
Fill the vial with 0.75 mL of formulation buffer and wash by inversion 10 times;
Empty the vial from washing solution and fill again with 0.75 mL of API;
Repeat steps 2) and 3) three times and empty the vial;
Fill the vial with 3.845 mL of Bio-Rad colloidal gold nanoparticle protein staining solution (#1706527);
Close the vial with stopper and capping;
Put the vial in a shaker (inside a 7×7 grid box);
NOTE: the vial needs to be in horizontal position during this step to avoid sedimentation of the staining gold nanoparticles and allow homogeneous coverage of the glass surface.
Incubate vials for 24 hours at room temperature with gentle shaking (35-45 rpm);
After step 8) is complete, remove all staining solution from the vial;
Wash the vial with Milli Q water by filling completely the vial and inverting three times.
Repeat step 10) three times; and
Remove Milli Q water completely, with help of pipette if required but gently in order not to scratch and damage the staining ribbon on the glass vial surface.

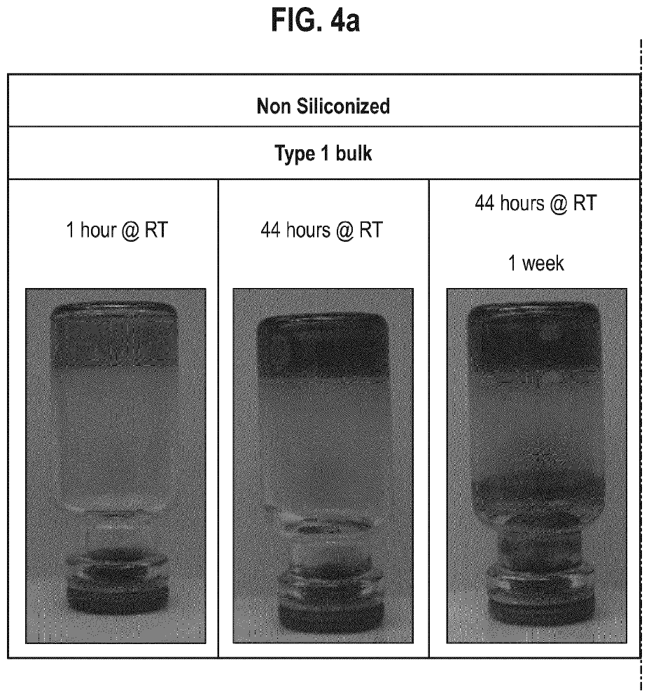

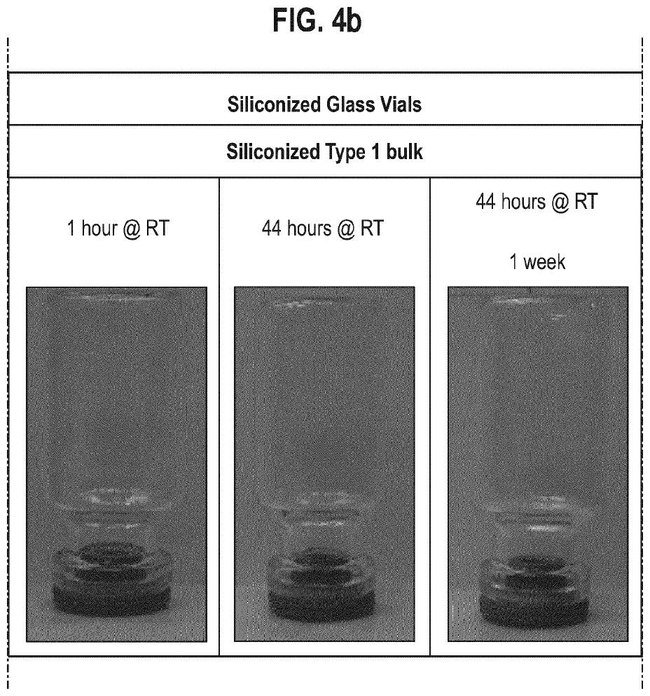

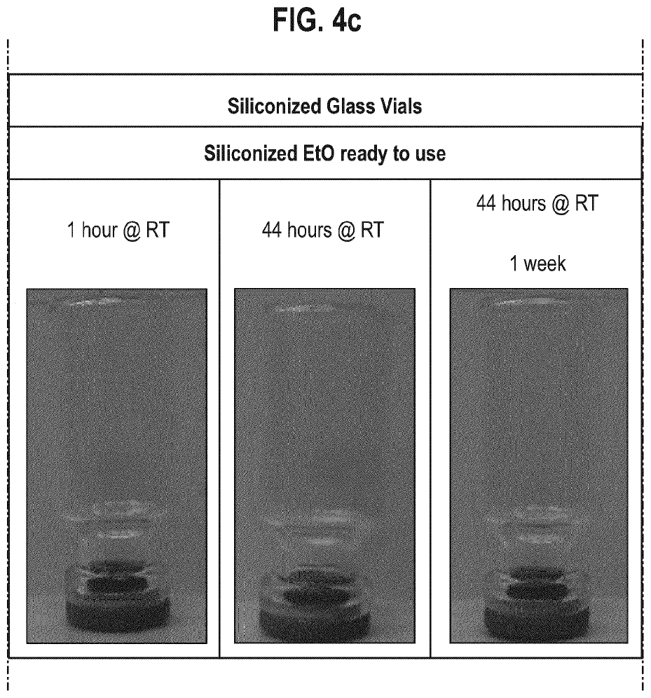

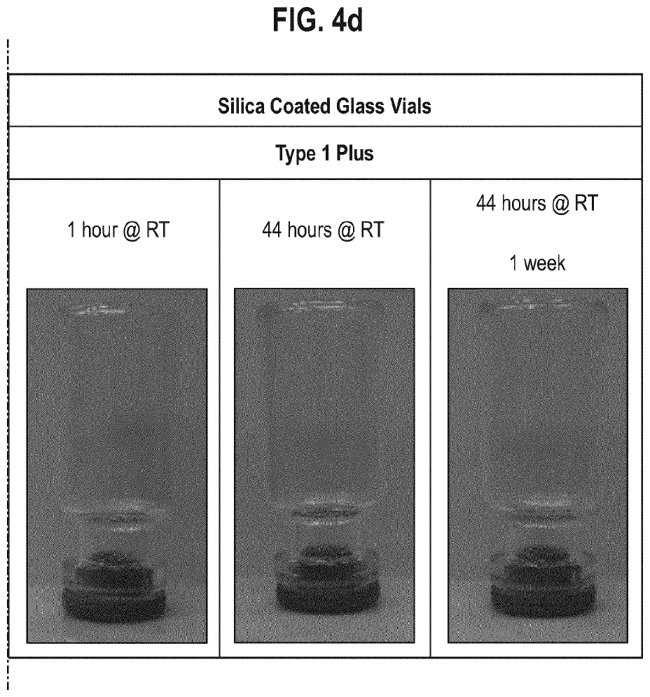

This process was performed for Type 1 standard vials Bulk, which have no coating on the internal surface, as well as siliconized (Baked-on) Bulk containers, and Siliconized (Baked-on) EZ-fill EtO containers from Nuova Ompi. Additionally, the process was carried out on the Type 1 Plus (SiO2) containers, which are Type 1 borosilicate vials with a quartz-like coating from Schott. FIGS. 4a-d demonstrate the obvious staining of the uncoated glass vial, after 1 hour and 44 hours at room temperature. A further sample was kept for 44 hours at room temperature and then inverted and stored for one week at 2-8° C. before being tested. This inverted sample clearly shows additional staining caused by the vaccine composition residing for extended periods in two distinct areas of the vial. This demonstrates that Ad26 adsoprtion had occurred where the vaccine composition came into contact with the internal surface of the glass vial. These results on the uncoated vials (FIG. 4a) can be contrasted with the results on the siliconized (FIGS. 4b and 4c) and SiO₂ coated (FIG. 4d) vials, where no staining was observed demonstrating a significant reduction in Ad26 adsorption.

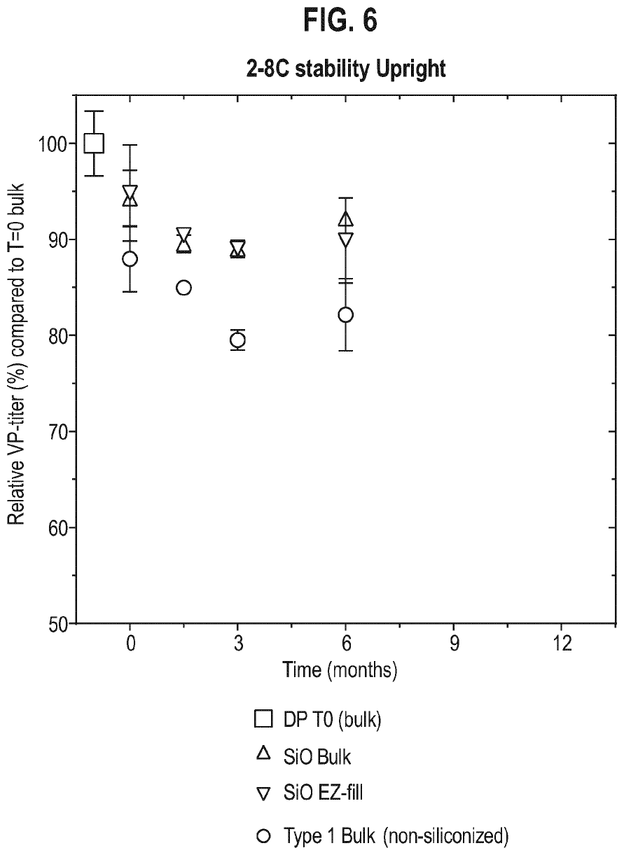

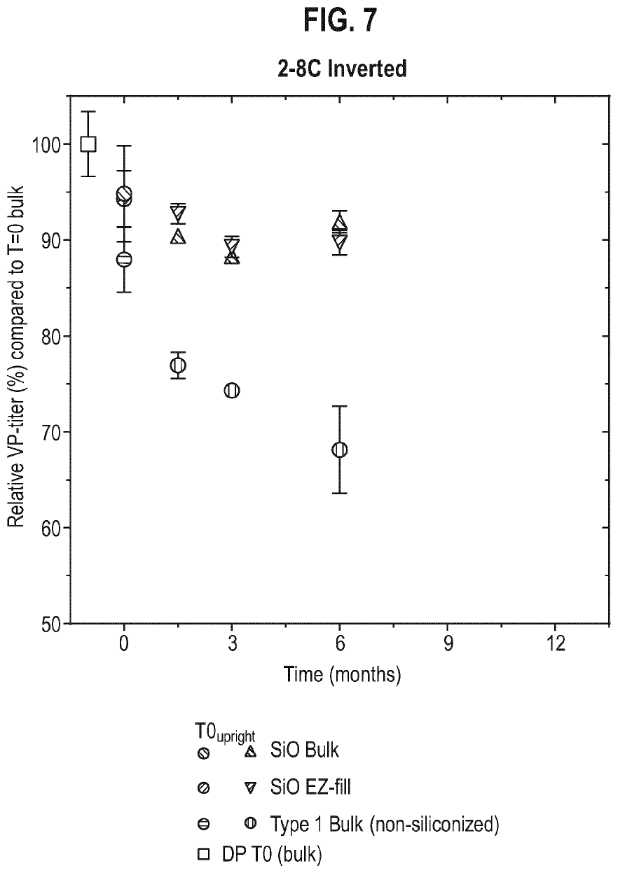

Longer term stability of Ad26 within siliconized vials is demonstrated by FIG. 6 and FIG. 7. These data compare Type 1 standard vials Bulk (Type 1 Bulk) to two siliconized vials: siliconized (Baked-on) Bulk containers (SiO Bulk) and the Siliconized (Baked-on) EZ-fill EtO containers (SiO EZ-fill) from Nuova Ompi. Drug substance was taken from storage (between −85° C. and −55° C.), compounded to a concentration of 2.0×1011 VP/mL in formulation buffer and filtered. The DP TO (bulk) sample was aliquoted directly in Eppendorf tubes from the filtered bulk solution ensuring no contact with any glass surfaces and the VP titer was determined using capilliary zone electrophoresis (CZE). The filtered drug product was also filled in the 3 different vial types with 0.75 mL per vial. After 44 h of holding time at 25° C. in upright orientation, the vials were placed in upright and inverted orientation to start long-term stability at 2-8° C. for up to six months. VP titer was again determined by CZE method and stability plots were plot relative to DP TO (bulk) sample. The results in FIG. 6 and FIG. 7 demonstrate the higher stability of samples in siliconized vials relative to standard Type 1 Bulk vials.

1.-15. (canceled)

16. A method of maintaining potency of a vaccine composition comprising virus particles, comprising:
providing a container that comprises an internal surface, the internal surface comprising at least one of (i) a polymeric material, (ii) silicon dioxide, or (iii) a surface treated with ethylene oxide; and
introducing the vaccine composition into the container and in contact with the internal surface.

17. A method of reducing loss of virus particles from a vaccine composition, comprising:
providing a container that comprises an internal surface, the internal surface comprising at least one of (i) a polymeric material, (ii) silicon dioxide, or (iii) a surface treated with ethylene oxide; and
introducing the vaccine composition into the container and in contact with the internal surface;
wherein the silicon dioxide or the polymeric material are present as part of the internal surface of the container and wherein the treatment with ethylene oxide is applied to an internal surface of the container.

18. A vaccine product comprising:
a container comprising an internal surface comprising at least one of: (i) a polymeric material, (ii) silicon dioxide, or (iii) a surface treated with ethylene oxide; and
a vaccine composition within the container in contact with the internal surface, wherein the vaccine composition comprises virus particles.

19. The method of claim 16, wherein the container comprises a borosilicate glass.

20. The method of claim 16, wherein the silicon dioxide or polymeric material is present as a coating.

21. The method of claim 16, wherein the silicon dioxide or polymeric material is present substantially throughout the internal surface of the container.

22. The method of claim 20, wherein the polymeric material is a polysiloxane.

23. The method of claim 16, wherein the container consists essentially of polypropylene.

24. The method of claim 16, wherein the container consists essentially of a cyclic olefin based resin.

25. The method of claim 16, wherein the vaccine composition comprises virus particles having an isoelectric point of from 6 to 8.

26. The method of claim 16, wherein the vaccine composition comprises virus particles having an isoelectric point value within 1 pH unit of the pH of the vaccine composition.

27. The method of claim 16, wherein the vaccine composition has a pH of about 7.

28. The method of claim 16, wherein the virus particles are adenovirus particles.

29. The method of claim 16, wherein the virus particles are inactivated poliovirus or attenuated poliovirus and the inactivated poliovirus or attenuated poliovirus comprises serotype 2, and wherein the inactivated poliovirus or attenuated poliovirus is a Sabin strain.
